(19) United States
(12) Patent Application Publication
(10) Pub. No.: US 2019/0298677 A1
(43) Pub. Date: Oct. 3, 2019

(54) Methods of Treating Amyotrophic Lateral Sclerosis and Symptoms

(71) Applicant: Steven Hoffman, Mahway, NJ (US)

(72) Inventor: Steven Hoffman, Mahway, NJ (US)

(21) Appl. No.: 16/437,702

(22) Filed: Jun. 11, 2019

Related U.S. Application Data
(63) Continuation of application No. 15/392,352, filed on Dec. 28, 2016, now abandoned.
(60) Provisional application No. 62/271,687, filed on Dec. 28, 2015.
(60) Provisional application No. 62/372,046, filed on Aug. 8, 2016.

(51) Int. Cl. A61K 31/198; A61K 31/575; A61K 31/402; A61K 31/167; A61K 45/06; A61K 31/426; A61K 31/216

(52) U.S. Cl. CPC A61K 31/198; A61K 31/575; A61K 31/402; A61K 31/216; A61K 45/06; A61K 31/426; A61K 31/167

(57) ABSTRACT

The present disclosure is directed to methods, compositions, and kits for treating amyotrophic lateral sclerosis.

### CROSS REFERENCE TO RELATED APPLICATIONS

This application is a continuation of U.S. application Ser. No. 15/392,352 filed Dec. 28, 2015 which claims the benefit of U.S. Provisional Application Nos. 62/271,687, filed Dec. 28, 2015 and 62/372,046, filed Aug. 8, 2016, the entireties of which are incorporated by reference herein.

### TECHNICAL FIELD

The present disclosure relates generally to compositions, kits, and methods for the treatment of amyotrophic lateral sclerosis and its symptoms.

### BACKGROUND

Amyotrophic lateral sclerosis (“ALS,” “Lou Gehrig's disease,” or “Charcot disease”) is a severely debilitating, progressive, neurodegenerative disease. In ALS, the upper and lower motor neurons in the motor cortex of the brain, brain stem, and spinal cord die, resulting in muscle weakness and atrophy throughout the body. The symptoms of ALS are numerous and can vary from patient to patient, but examples of the more commons symptoms include stiff muscles, muscle weakness, muscle wasting, difficulty speaking, difficulty swallowing, difficulty breathing, difficulty chewing, difficulty walking, fasciculations, and cramps.

There is no cure for ALS. Current treatments protocols focus on managing the symptoms of the disease, for example, with ventilator assistance, physical therapy, occupational therapy, and pain control. As such, new therapies to treat ALS are needed.

### SUMMARY

The present disclosure is directed to methods of treating amyotrophic lateral sclerosis (“ALS”) in a patient comprising administering to the patient an effective amount of a tyrosine hydroxylase inhibitor. Kits and pharmaceutical compositions for use in these methods are also described.

### DETAILED DESCRIPTION OF ILLUSTRATIVE EMBODIMENTS

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative, or palliative treatment. As used herein, the term “treating” includes alleviating or reducing at least one adverse or negative effect or symptom of ALS.

As employed above and throughout the disclosure the term “effective amount” refers to an amount effective, at dosages, and for periods of time necessary, to achieve the desired result with respect to the treatment of the relevant disorder, condition, symptom, or side effect. It will be appreciated that the effective amount of components of the present disclosure will vary from patient to patient not only with respect to the particular compound, component or composition selected, the route of administration, and the ability of the components to elicit a desired result in the individual, but also with respect to factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide improved therapeutic response. An effective amount is also one in which any toxic or detrimental effects of the components are outweighed by the therapeutically beneficial effects.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein can be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

The term “stereoisomers” refers to compounds that have identical chemical constitution, but differ as regards the arrangement of the atoms or groups in space. The term “enantiomers” refers to stereoisomers that are mirror images of each other that are non-superimposable.

The term “administering” means either directly administering a compound or composition of the present invention, or administering a prodrug, derivative or analog which will form an equivalent amount of the active compound or substance within the body.

The terms “subject,” “individual,” and “patient” are used interchangeably herein, and refer to an animal, for example a human, to whom treatment, including prophylactic treatment, with the pharmaceutical composition according to the present invention, is provided.

The term “inhibitor” as used herein includes compounds that inhibit the expression or activity of a protein, polypeptide or enzyme and does not necessarily mean complete inhibition of expression and/or activity. Rather, the inhibition includes inhibition of the expression and/or activity of a protein, polypeptide or enzyme to an extent, and for a time, sufficient to produce the desired effect.

The present disclosure is directed to methods of treating ALS in a patient by administering to the patient an effective amount of a tyrosine hydroxylase inhibitor. According to the disclosure, these methods include the treatment of ALS per se, as well as methods of treating one or more of the symptoms of ALS. As those skilled in the art will readily appreciate, the symptoms of ALS are numerous and vary from patient to patient. Those more common symptoms of ALS include stiff muscles, muscle weakness, muscle wasting, difficulty speaking, difficulty swallowing, difficulty breathing, difficulty chewing, difficulty walking, fasciculations, cramps, and any combination of these symptoms. In preferred embodiments, the methods of the disclosure are used to treat the muscle wasting symptoms of ALS. In some embodiments, the methods of the disclosure are used to treat the stiff muscle symptoms of ALS. In other embodiments, the methods of the disclosure are used to treat the muscle weakness symptoms of ALS. In other embodiments, the methods of the disclosure are used to treat the difficulty speaking symptoms of ALS. In other embodiments, the methods of the disclosure are used to treat the difficulty speaking symptoms of ALS. In other embodiments, the methods of the disclosure are used to treat the difficulty swallowing symptoms of ALS. In other embodiments, the methods of the disclosure are used to treat the difficulty breathing symptoms of ALS. In other embodiments, the methods of the disclosure are used to treat the difficulty chewing symptoms of ALS. In other embodiments, the methods of the disclosure are used to treat the difficulty walking symptoms of ALS. In other embodiments, the methods of the disclosure are used to treat the fasciculation symptoms of ALS. In other embodiments, the methods of the disclosure are used to treat the cramping symptoms of ALS.

Other aspects of the disclosure are directed to methods of treating ALS in a patient by administering to the patient an effective amount of a tyrosine hydroxylase inhibitor and an effective amount of a bile regulator. As used herein “bile regulators” refers to any compound that alters (i.e., elevates or decreases) circulating levels of bile acids or bile salts in a patient. Preferred bile regulators include bile acids or pharmaceutically acceptable salts thereof, farnesoid X receptor (FXR) agonists, liver X receptor (LXR) agonist, and peroxisome proliferator-activated receptor (PPAR) agonists. One or any combination of bile regulators can be used in the methods of the disclosure.

“Bile acids” are steroid acids predominantly found in the bile of mammals. Primary bile acids are synthesized by the liver; secondary bile acids are produced from bacterial action in the colon. Preferred bile acids or pharmaceutically acceptable salts thereof include, for example, ursodeoxycholic acid, cholic acid, chenodeoxycholic acid (also referred to as chenocholic acid), taurochenodeoxycholic acid, lithocholic acid, taurocholic acid, glycocholic acid, deoxycholic acid, and glycochenodeoxycholic acid, as well as combinations thereof. In general, a typical daily dose of the bile acid or pharmaceutically acceptable salt thereof, is in the range of from about 0.01 mg/kg to about 100 mg/kg. This dose may be administered as a single unit dose or as several separate unit doses.

Preferred bile regulators for the treatment of ALS are those that decrease circulating levels of bile acids and/or bile salts in a patient. Representative bile regulators of this type include: somatostatin; cortistatin; precortistatin; octreotide, bosentan, chlorpromazine, clofazimine, cyclosporine, ethinyl estradiol, fusidic acid, glyburide, ketoconazole, novobiocin, paclitaxel, ponatinib, progesterone, quinidine, reserpine, rifampicin, tamoxifen, troglitazone, ursodeoxycholic acid, verapamil, vinblastine; fibroblast growth factor 19 (FGF19); estrogen blockers such as tamoxifen, arimidex, letrozole, and raloxifene; bile salt sequestrants such as colestyramine, colestipol, and colesevelam; proton pump inhibitors (PPIs) such as omeprazole, lansoprazole, dexlansoprazole, esomeprazole, pantoprazole, rabeprazole, and ilaprazole; H2 blockers such as cimetidine, famotidine, nizatidine, and ranitidine; antacids such as aluminum hydroxide, calcium carbonate, magnesium carbonate, magnesium hydroxide, sodium bicarbonate, sodium carbonate; and combinations of any of the foregoing. Without being bound to any particular theory, it is believed that ALS may be effectively treated through administration of one or more compounds that decrease circulating levels of bile acids and/or bile salts in a patient.

Farnesoid X receptor (FXR) is also known as the bile acid receptor (“BAR”). Certain bile acids can be FXR agonists. Non-bile acid FXR agonists, and pharmaceutically acceptable salts thereof, are also useful in the methods of the disclosure. An FXR agonist activates FXR at a concentration below 1 μM, below 0.8 μM, below 0.6 μM, below 0.4 μM, or below 0.2 (e.g., as measured by an AlphaScreen assay). FXR agonists are known in the art and include, for example, GW4064 (CAS No. 278779-30-9), cafestol, and fexaramine. Obeticholic acid (also referred to as 6-α-ethyl-chenodeoxycholic acid and INT-747) is a semi-synthetic bile acid analogue that is an FXR agonist that is also useful in the methods of the disclosure. See also, Neuschwander-Teti, B A, Curr. Gastroenteral. Rep. 2012 February; 14 (1):55-56; U.S. Pat. No. 8,969,330; U.S. Published Application No. 2014/0371190. In general, a typical daily dose of the FXR agonist is in the range of from about 0.01 mg/kg to about 100 mg/kg. This dose may be administered as a single unit dose or as several separate unit doses.

Liver X receptor (LXR) is a member of the nuclear receptor family of transcription factors. LXR agonists, and pharmaceutically acceptable salts thereof, are known in the art and include, for example, hypocholamide (3α, 6α-dihydroxy-5β-cholanoic acid-N-methyl-N-methoxy-24-amide), T0901317 (CAS No. 293754-55-9), GW3965 (CAS No. 405911-17-3), N,N-dimethyl-3beta-hydroxy-cholenamide (DMHCA), and WAY-252623 (CAS No. 875787-07-8). An LXR agonist activates LXR at a concentration below 1 μM, below 0.8 μM, below 0.6 μM, below 0.4 μM, or below 0.2 (e.g., as measured by an AlphaScreen assay). In general, a typical daily dose of the LXR agonist is in the range of from about 0.01 mg/kg to about 100 mg/kg. This dose may be administered as a single unit dose or as several separate unit doses.

Peroxisome Proliferator-Activated Receptors (PPAR) are involved in lipid regulation and homeostasis. PPAR-α agonists have been investigated for use in the treatment of cholesterol disorders associated with high triglycerides. PPAR-α agonists useful in the methods of the disclosure include fibrate drugs, for example, clofibrate, gemfibrozil, ciprofibrate, bezafibrate, and fenofibrate. PPAR-γ agonists have been investigated for use in the treatment of diabetes and atherosclerosis. PPAR-γ agonists useful in the methods of the disclosure include, for example, non-steroidal anti-inflammatory drugs (e.g., ibuprofen) and thiazolidinediones (TZDs) such as GW-9662. PPAR-δ agonists have been investigated for use in balancing glucose and lipid oxidation and the regulation of how the body uses fuels. PPAR-δ agonists that are useful in the methods of the disclosure include, for example, GW501516. Dual PPAR agonists are also known in the art. Dual PPAR agonists useful in the methods of the disclosure include, for example, aleglitazar, muraglitazar, tesaglitazar, and saroglitazar. A PPAR agonist will activate a PPAR at a concentration below 1 μM, below 0.8 μM, below 0.6 μM, below 0.4 μM, or below 0.2 (e.g., as measured by an AlphaScreen assay). In general, a typical daily dose of the PPAR agonist is in the range of from about 0.01 mg/kg to about 100 mg/kg. This dose may be administered as a single unit dose or as several separate unit doses

While preferred administered amounts of the bile regulators have been described herein, one skilled in the art will understand that the amount of bile regulator effective to treat ALS and/or the symptoms of ALS can also be determined by one skilled in the art.

In certain embodiments, the tyrosine hydroxylase inhibitor is a tyrosine derivative. The tyrosine derivative can be capable of existing in different isomeric forms, including stereoisomers and enantiomers. The tyrosine derivative can, for example, exist in both L-form or D-form. The tyrosine derivative can, for example, also exist in a racemic form. Representative tyrosine derivatives include one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-tyrosine(tBu)-allyl ester hydrochloride, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6- fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyrosine methyl ester hydrochloride, H-3,5-diiodo-tyrosine methyl ester hydrochloride, H-D-3,5-diiodo-tyrosine methyl ester hydrochloride, H-D-tyrosine methyl ester hydrochloride, D-tyrosine methyl ester hydrochloride, D-tyrosine-methyl ester hydrochloride, methyl D-tyrosinate hydrochloride, H-D-tyrosine methyl ester·hydrochloride, D-tyrosine methyl ester hydrochloride, H-D-tyrosine methyl ester-hydrochloride, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-tyrosine (3,5-I2)-OSu, Fmoc-tyrosine(3-NO2)-OH, α-methyl-L-tyrosine, α-methyl-D-tyrosine, and α-methyl-DL-tyrosine. In certain embodiments of the invention, the tyrosine derivative is α-methyl-L-tyrosine. In other embodiments, the tyrosine derivative is α-methyl-D-tyrosine. In other embodiments, the tyrosine derivative is α-methyl-DL-tyrosine in a racemic form.

In general, a typical daily dose of the tyrosine hydroxylase inhibitor is in the range of from about 0.01 mg/kg to about 100 mg/kg. In some aspects, between about 50 mg and 300 mg or between about 75 mg and 150 mg or about 100 mg of the tyrosine hydroxylase inhibitor, preferably as a tyrosine derivative such as α-methyl-L-tyrosine, α-methyl-D-tyrosine, or α-methyl-DL-tyrosine, is administered daily. This dose may be administered as a single unit dose or as several separate unit doses. In preferred aspects, the tyrosine hydroxylase inhibitor, preferably as a tyrosine derivative such as α-methyl-L-tyrosine, α-methyl-D-tyrosine, or α-methyl-DL-tyrosine, is administered in three or four, preferably three, substantially equal doses, each day.

The administration of the tyrosine hydroxylase inhibitor can be through various routes, including orally, nasally, subcutaneously, intravenously, intramuscularly, transdermally, vaginally, rectally, or in any combination thereof. Transdermal administration can be effected using, for example, oleic acid, 1-methyl-2-pyrrolidone, dodecylnonaoxyethylene glycol monoether.

In other embodiments employing the administration of a bile regulator in addition to the tyrosine hydroxylase inhibitor, the bile regulator can be administered through various routes, including orally, nasally, subcutaneously, intravenously, intramuscularly, transdermally, vaginally, rectally, or in any combination thereof. Transdermal administration can be effected using, for example, oleic acid, 1-methyl-2-pyrrolidone, dodecylnonaoxyethylene glycol monoether.

In some embodiments, the bile regulator and tyrosine hydroxylase inhibitor are administered concurrently. In other embodiments, the bile regulator and tyrosine hydroxylase inhibitor are administered sequentially.

The tyrosine hydroxylase inhibitor can be administered during a cycle consisting daily dosing for 30 days as a single cycle. In other aspects, the tyrosine hydroxylase inhibitor is administered in divided doses.

The bile regulator can be administered during a cycle consisting of five to seven days of administering the bile regulator, and one to two days of not administering the bile regulator. The bile regulator can be administered over the course of at least six said cycles. In one suitable embodiment of the invention, the bile regulator is administered daily. In another suitable embodiment of the invention, the bile regulator is administered multiple times per day.

According to the described methods, the patient's ALS progression and/or symptoms are assessed. This assessment can occur prior to initiating treatment with the bile regulator and tyrosine hydroxylase inhibitor. This assessment can alternatively or also occur during the course of the treatment period with the bile regulator and the tyrosine hydroxylase inhibitor.

Also included within the scope of the disclosure are kits useful for practicing the described methods. The kits comprise at least one bile regulator, i.e., a bile acid or a pharmaceutically acceptable salt thereof, an FXR agonist, an LXR agonist, a PPAR agonist, or a combination thereof; and a tyrosine hydroxylase inhibitor; together with packaging and instructions for using the kit to treat ALS.

The disclosure is also directed to pharmaceutical compositions for use in the described methods. These compositions include a bile regulator, a bile acid or a pharmaceutically acceptable salt thereof; an FXR agonist, an LXR agonist, a PPAR agonist, or a combination thereof; a tyrosine hydroxylase inhibitor; and a pharmaceutically acceptable excipient. In some aspects, the pharmaceutical compositions for use in the described methods comprise a bile acid or a pharmaceutically acceptable salt thereof; a tyrosine hydroxylase inhibitor; and a pharmaceutically acceptable excipient. In other aspects, the pharmaceutical compositions for use in the described methods comprise an FXR agonist, a tyrosine hydroxylase inhibitor, and a pharmaceutically acceptable excipient. In other aspects, the pharmaceutical compositions for use in the described methods comprises an LXR agonist, a tyrosine hydroxylase inhibitor, and a pharmaceutically acceptable excipient. In still other aspects, the pharmaceutical compositions for use in the described methods comprise a PPAR agonist, a tyrosine hydroxylase inhibitor, and a pharmaceutically acceptable excipient. Pharmaceutically acceptable excipient(s) include but are not limited to diluents, lubricants, disintegrants, glidants, and surface-active agents.

The disclosure is also directed to methods of treating ALS by administering to the patient an effective amount of a tyrosine hydroxylase inhibitor and an effective amount of another pharmaceutical composition for use in the treatment of ALS or ALS symptoms. Other embodiments are directed to methods of treating ALS by administering to the patient an effective amount of a tyrosine hydroxylase inhibitor, and effective amount of a bile regulator, and an effective amount of another pharmaceutical composition for use in the treatment of ALS or ALS symptoms. Other pharmaceutical compositions useful for treating ALS or ALS symptoms include, for example, riluzole, baclogen, diazepam, trihexyphenidyl, and amitriptyline. Other pharmaceutical compositions useful for treating ALS or ALS symptoms include those used for treating, for example, muscle cramps and spasms, spasticity, constipation, fatigue, excessive salivation, excessive phlegm, pain, depression, sleep problems, uncontrolled outburst of laughing or crying, and breathing problems.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### EXAMPLES

Patients having a diagnosis of ALS are screened and their symptoms assessed. A first subgroup of those patients is administered a treatment regimen that includes a tyrosine hydroxylase inhibitor (i.e., α-methyl-DL tyrosine) at dose of 50-300 mg daily, either as a single dose or as divided doses.

A second subgroup of the patient is administered a treatment regimen that includes a tyrosine hydroxylase inhibitor (i.e., α-methyl-DL tyrosine) at dose of 50-300 mg daily, either as a single dose or as divided doses, and a bile regulator (i.e., a bile acid such as cholic acid) at a dose of 25-500 mg daily, either as a single dose or as divided doses.

A third subgroup of the patient is administered a treatment regimen that includes a tyrosine hydroxylase inhibitor (i.e., α-methyl-DL tyrosine) at dose of 50-300 mg daily, either as a single dose or as divided doses, and a bile regulator (i.e., an FXR agonist) at a dose of 0.01 mg/kg to about 100 mg/kg daily, either as a single dose or as divided doses.

A fourth subgroup of the patient is administered a treatment regimen that includes a tyrosine hydroxylase inhibitor (i.e., α-methyl-DL tyrosine) at dose of 50-300 mg daily, either as a single dose or as divided doses, and a bile regulator (i.e., an LXR agonist) at a dose of 0.01 mg/kg to about 100 mg/kg daily, either as a single dose or as divided doses.

A fifth subgroup of the patient is administered a treatment regimen that includes a tyrosine hydroxylase inhibitor (i.e., α-methyl-DL tyrosine) at dose of 50-300 mg daily, either as a single dose or as divided doses, and a bile regulator (i.e., an PPAR agonist) at a dose of 0.01 mg/kg to about 100 mg/kg, either as a single dose or as divided doses.

For a study period of four weeks, for each subgroup, changes in ALS symptoms are re-assessed following administration of the treatment regimen.

What is claimed:

1. A method of treating amyotrophic lateral sclerosis in a patient comprising administering to the patient an effective amount of a tyrosine hydroxylase inhibitor.

2. The method of claim 1, further comprising administering to the patient an effective amount of a bile regulator.

3. The method of claim 2, wherein the bile regulator is a bile acid or a pharmaceutically acceptable salt thereof, an FXR agonist, an LXR agonist, a PPAR agonist, or a combination thereof.

4. The method of claim 1 wherein the treatment comprises reducing at least one symptom of amyotrophic lateral sclerosis.

5. The method of claim 4, wherein the symptom of amyotrophic lateral sclerosis is stiff muscles, muscle weakness, muscle wasting, difficulty speaking, difficulty swallowing, difficulty breathing, difficulty chewing, difficulty walking, fasciculations, cramps, or any combination thereof.

6. The method of claim 4, wherein the symptom of amyotrophic lateral sclerosis is muscle wasting.

7. The method of claim 1, wherein the tyrosine hydroxylase inhibitor is administered orally, subcutaneously, intravenously, transdermally, vaginally, rectally or in any combination thereof.

8. The method of claim 2, wherein the bile regulator is administered orally, subcutaneously, intravenously, transdermally, vaginally, rectally or in any combination thereof.

9. The method of claim 2, wherein the bile regulator and the tyrosine hydroxylase inhibitor are administered concurrently or sequentially.

10. The method of claim 2, wherein the bile regulator is a bile acid or pharmaceutically acceptable salt thereof.

11. The method of claim 10, wherein the bile acid is ursodeoxycholic acid, cholic acid, chenodeoxycholic acid (also referred to as chenocholic acid), taurochenodeoxycholic acid, lithocholic acid, taurocholic acid, glycocholic acid, deoxycholic acid, or glycochenodeoxycholic acid, or a pharmaceutically acceptable salt thereof, or a combination thereof.

12. The method of claim 2, wherein the bile regulator is a farnesoid X receptor (FXR) agonist.

13. The method of claim 12, wherein the FXR agonist is GW4064, cafestol, fexaramine, obeticholic acid, or a pharmaceutically acceptable salt thereof, or a combination thereof

14. The method claim 2, wherein the bile regulator is a liver X receptor (LXR) agonist.

15. The method of claim 14, wherein the LXR agonist is hypocholamide, T0901317, GW3965, N,N-dimethyl-3beta-hydroxy-cholenamide (DMHCA), or WAY-252623, or a pharmaceutically acceptable salt thereof, or a combination thereof.

16. The method of claim 2, wherein the bile regulator is a peroxisome proliferator-activated receptor (PPAR) agonist.

17. The method of claim 16, wherein the PPAR agonist is a PPAR-α agonist, a PPAR-γ agonist, a PPAR-δ agonist, or a combination thereof.

18. The method of claim 16, wherein the PPAR agonist is a fibrate drug or a thiazolidinedione (TZD).

19. The method of claim 18, wherein the fibrate drug is clofibrate, gemfibrozil, ciprofibrate, bezafibrate, or fenofibrate.

20. The method of claim 18 wherein the TZD is GW-9662.

21. The method of claim 16, wherein the PPAR agonist is GW501516.

22. The method of claim 16, wherein the PPAR agonist is aleglitazar, muraglitazar, tesaglitazar, or saroglitazar.

23. The method of claim 2, wherein the bile regulator is a combination of any of the bile acid or pharmaceutically acceptable salt thereof; the FXR agonist; the LXR agonist, and the PPAR agonist.

24. The method of claim 1, wherein the tyrosine hydroxylase inhibitor is a tyrosine derivative.

25. The method of claim 24 wherein the tyrosine derivative is one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyr-OME HCl, H-3,5-diiodo-tyr-OME HCl, H-D-3,5-diiodo-tyr-OME HCl, H-D-tyr-OME HCl, D-tyrosine methyl ester hydrochloride, D-tyrosine-ome HCl, methyl D-tyrosinate hydrochloride, H-D-tyr-OMe.HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I₂)-OSu, Fmoc-tyr(3-NO₂)-OH, α-methyl-L-tyrosine, α-methyl-D-tyrosine, and α-methyl-DL-tyrosine.

26. The method of claim 24 wherein the tyrosine derivative is α-methyl-L-tyrosine.

27. The method of claim 24 wherein the tyrosine derivative is α-methyl-D-tyrosine.

28. The method of claim 24 wherein the tyrosine derivative is racemic α-methyl-DL-tyrosine.

29. The method of claim 24 wherein 150-300 mg of the tyrosine derivative is administered daily.

30. The method of claim 29, wherein the tyrosine derivative is administered in three substantially equal doses, each day.

31. The method of claim 29, wherein the tyrosine derivative is administered in four substantially equal doses, each day.

32. The method of claim 1, further comprising administering to the patient an effective amount of another pharmaceutical composition useful in the treatment of amyotrophic lateral sclerosis or a symptom of amyotrophic lateral sclerosis.

33. The method of claim 32, wherein the other pharmaceutical composition is riluzole, baclogen, diazepam, trihexyphenidyl, or amitriptyline.

34. The method of claim 32, wherein the other pharmaceutical composition is for the treatment of muscle cramps, muscle spasms, spasticity, constipation, fatigue, excessive salivation, excessive phlegm, pain, depression, sleep problems, uncontrolled outburst of laughing or crying, or breathing problems

35. A pharmaceutical composition comprising an effective amount of a tyrosine hydroxylase inhibitor; an effective amount of a bile acid or a pharmaceutically acceptable salt thereof; an FXR agonist; an LXR agonist; a PPAR agonist; or a combination thereof; and a pharmaceutically acceptable excipient.

36. The method of claim 2, wherein the bile regulator is somatostatin; cortistatin, precortistatin, octreotide, bosentan, chlorpromazine, clofazimine, cyclosporine, ethinyl estradiol, fusidic acid, glyburide, ketoconazole, novobiocin, paclitaxel, ponatinib, progesterone, quinidine, reserpine, rifampicin, tamoxifen, troglitazone, ursodeoxycholic acid, verapamil, vinblastine; fibroblast growth factor 19, tamoxifen, arimidex, letrozole, raloxifene, colestyramine, colestipol, colesevelam, omeprazole, lansoprazole, dexlansoprazole, esomeprazole, pantoprazole, rabeprazole, ilaprazole, cimetidine, famotidine, nizatidine, ranitidine, aluminum hydroxide, calcium carbonate, magnesium carbonate, magnesium hydroxide, sodium bicarbonate, sodium carbonate, or a combination of any of the foregoing.

37. A method of treating amyotrophic lateral sclerosis in a patient comprising administering to the patient an effective amount of bile regulator that decreases the circulating levels of bile acids and/or bile salts.

38. The method of claim 37 wherein the bile regulator is somatostatin; cortistatin, precortistatin, octreotide, bosentan, chlorpromazine, clofazimine, cyclosporine, ethinyl estradiol, fusidic acid, glyburide, ketoconazole, novobiocin, paclitaxel, ponatinib, progesterone, quinidine, reserpine, rifampicin, tamoxifen, troglitazone, ursodeoxycholic acid, verapamil, vinblastine; fibroblast growth factor 19, tamoxifen, arimidex, letrozole, raloxifene, colestyramine, colestipol, colesevelam, omeprazole, lansoprazole, dexlansoprazole, esomeprazole, pantoprazole, rabeprazole, ilaprazole, cimetidine, famotidine, nizatidine, ranitidine, aluminum hydroxide, calcium carbonate, magnesium carbonate, magnesium hydroxide, sodium bicarbonate, sodium carbonate, or a combination of any of the foregoing.
